(19) United States
(12) Patent Application Publication
(10) Pub. No.: US 2022/0357329 A1
(43) Pub. Date: Nov. 10, 2022

(54) PREDICTIVE LIQUID MARKERS FOR CANCER IMMUNOTHERAPY

(71) Applicant: Accure Health Inc., Allston, MS (US)

(72) Inventors: Liyun Sang, Allston, MA (US); Qiang Liu, Allston, MA (US)

(21) Appl. No.: 17/622,611

(22) PCT Filed: Jul. 1, 2020
(86) PCT No.: PCT/US20/40458

§ 371 Date: Dec. 23, 2021

Related U.S. Application Data
(60) Provisional application No. 62/869,290, filed on Jul. 1, 2019.

(51) Int. Cl. G01N 33/574

(52) U.S. Cl. CPC G01N 33/57488; G01N 2333/70596; G01N 2333/47; G01N 2333/575; G01N 2333/916; G01N 2333/70532; G01N 2333/70521; G01N 2333/70503; G01N 2333/5434

(57) ABSTRACT

The present disclosure relates generally to methods and compositions for cancer immunotherapy, and more specifically, liquid markers for predicting effectiveness of cancer therapies. The disclosure features compositions and methods that are useful for predicting the efficacy of cancer treatment (e.g., a checkpoint inhibitor immunotherapy) and, in some embodiments, administering the cancer treatment such as immunotherapy.

### CROSS-REFERENCE TO RELATED APPLICATIONS

This application claims priority to and the benefit of U.S. Provisional Patent Application No. 62/869,290 filed Jul. 1, 2019, the entire disclosure of which is incorporated herein by reference.

### FIELD

The present disclosure relates generally to methods and compositions for cancer immunotherapy and tumor microenvironment (TME), and more specifically, liquid markers for predicting effectiveness of checkpoint inhibitor immunotherapy.

### BACKGROUND

Immunotherapy is gaining momentum as an effective therapeutic approach in a variety of cancers. However, despite the clinical success of the first wave of immune checkpoint inhibitors (CTLA4, PD-1 and PD-L1), only a subset of unselected patients exhibits durable responses. The number of immune-oncology targets is high and growing and the number of potential combinations of therapeutic agents directed against these targets and of combinations of such agents with conventional standard-of-care agents is even greater. Therefore, novel biomarkers are needed to guide the choice of these agents to obtain the maximal likelihood of patient benefit.

Predictive biomarkers for immunotherapy differ from the traditional biomarkers used for targeted therapies: the complexity of the immune response and tumor biology requires a more holistic approach than the use of a single analyte biomarker. Existing immunotherapy biomarker approaches include measuring PD-1/PD-L1 expression in cells (both immune cells and tumor cells in cancer), evaluating tumor mutation burden (TMB), testing microsatellite instability (MSI), detecting mismatch repair deficiency (MMR), and characterizing T cell-tumor microenvironment. Most of these approaches are tissue-based immunohistochemistry (IHC) or sequencing tests. A few liquid biopsy approaches for immunotherapy are currently under investigation, including imaging analyses of circulating tumor cells (CTC) and sequencing analyses of circulating tumor DNA (ctDNA). Extracellular biomarkers for immunotherapy have been largely unexplored. Many proteins involved in mismatch repair deficiency (MMR proteins), tumor microenvironment, or tumorigenesis are either nuclear or cytoplasmic proteins. The presence of these proteins in cell-free or extracellular locations were unforeseen

Thus, a need exists for non-invasive and inexpensive techniques to accurately characterize a cancer phenotype and tumor microenvironment, and predict the effectiveness of available therapeutic options. To that end, extracellular biomarkers for cancer immunotherapy are needed.

### SUMMARY

As described herein, the disclosure features compositions and methods that are useful for predicting the efficacy of cancer treatment (e.g., a checkpoint inhibitor immunotherapy) and, in some embodiments, administering the cancer treatment such as immunotherapy. Also described herein are methods for detecting and characterizing cancer-related markers associated with extracellular vesicles (“EVs”). In some embodiments, such markers can be present within the EVs (e.g., inside the phospholipid layer) or on the surface of the EV phospholipid layer.

In one aspect, a method for predicting effectiveness of checkpoint inhibitor immunotherapy is provided. The method can include: providing a bodily fluid sample from a subject, wherein the bodily fluid sample comprises extracellular vesicles; and determining an amount of one or more biomarkers that are associated with the extracellular vesicles, in comparison with a control from a healthy subject, wherein the biomarkers are selected from: MLH1, MSH2, MSH6, PMS2, Perforin, Granzymes, IL-2, IL-6, IL-12, TCF7, and Lin28, and optionally one or more of PD-L1, CD47, and PD-1.

In another aspect, a method for cancer immunotherapy is provided, comprising: providing a bodily fluid sample from a subject, wherein the bodily fluid sample comprises extracellular vesicles; determining an amount of one or more biomarkers that are associated with the extracellular vesicles, in comparison with a control from a healthy subject, wherein the biomarkers are selected from: MLH1, MSH2, MSH6, PMS2, Perforin, Granzymes, IL-2, IL-6, IL-12, TCF7, and Lin28, and optionally one or more of PD-L1, CD47, and PD-1; and administering a checkpoint inhibitor immunotherapy to the subject.

A further aspect relates to a method for diagnosing or prognosing cancer, comprising: providing a bodily fluid sample from a subject, wherein the bodily fluid sample comprises extracellular vesicles; and determining an elevated amount of a biomarkers that are associated with the extracellular vesicles, in comparison with a control, wherein the biomarker is Lin28 and optionally one or more of PD-L1, CD47, and PD-1.

In one embodiment, the biomarkers are MLH1, MSH2, PD-L1, CD47, and Lin28.

In one embodiment, the biomarkers are MLH1, MSH2, PD-L1, CD47, and IL-12.

In one embodiment, the biomarkers are MLH1, MSH2, PD-L1, CD47, and TCF7.

In one embodiment, the biomarkers are MLH1, MSH2, PD-L1, TCF7, and Lin28.

In one embodiment, the biomarkers are MLH1, MSH2, PD-L1, IL-12, and Lin28.

In one embodiment, the biomarkers are MLH1, MSH2, PD-L1, IL-12 and TCF7.

In one embodiment, the biomarkers are MLH1, MSH2, IL-12, TCF7 and Lin28.

In one embodiment, the biomarkers are PD-L1, CD47, IL-12, TCF7, and Lin28.

In one embodiment, the biomarkers are MLH1, MSH2, PD-L1, CD47, TCF7, and Lin28.

In one embodiment, the biomarkers are MLH1, MSH2, PD-L1, CD47, TCF7, and IL-12.

In one embodiment, the biomarkers are MLH1, MSH2, PD-L1, CD47, TCF7, IL-12, and Lin28.

In one embodiment, the biomarkers are MLH1, MSH2, PD-L1, Perforin, Granzymes, IL-12, and TCF7.

In one embodiment, the biomarkers are MLH1, MSH2, PD-L1, Perforin, Granzymes, IL-12, TCF7, and Lin28.

In one embodiment, the biomarkers are MLH1, MSH2, PD-L1, CD47, Granzymes, IL-12, TCF7, and Lin28.

In one embodiment, the biomarkers are MLH1, MSH2, PD-L1, CD47, Perforin, IL-12, TCF7, and Lin28.

In one embodiment, the biomarkers are MLH1, MSH2, PD-L1, CD47, Perforin, Granzymes, TCF7, and Lin28.

In one embodiment, the biomarkers are MLH1, MSH2, PD-L1, CD47, Perforin, Granzymes, IL-12, TCF7, and Lin28.

In one embodiment, the biomarkers are MLH1, MSH2, MSH6, PMS2, PD-L1, CD47, PD-1, Perforin, Granzymes, IL-2, IL-6, IL-12, TCF7, and Lin28.

In various embodiments, the bodily fluid sample can be plasma and/or urine.

In some embodiments, the determining step comprises using an integrated magnetic-electrochemical sensing (iMES) system. The determining step can include a SingleMarkerScore method, a CategoryScore method, a RegressionScore method, a DecisionTreeScore method, or a NeuralNetworkScore method.

In certain embodiments, the checkpoint inhibitor immunotherapy includes PD-1/PD-L1, CTLA4, and/or CD47 blockade.

In various embodiments, the cancer is one or more of: bladder, gastric cancer, lung cancer, colon cancer, prostate cancer, renal cell carcinoma, head and neck cancer, melanoma, liver cancer, esophageal cancer, pancreatic cancer, acute myeloid leukemia (AML), chronic myeloid leukemia (CML), glioblastoma, medulloblastoma, oral squamous cell carcinoma, breast cancer, ovarian cancer, cervical cancer, testicular germ cell tumor, and Wilms tumor.

In certain embodiments, the patient with cancer can be diagnosed or suspected of an additional disease, such as Covid-19.

The disclosure provides compositions and methods that are useful for predicting the efficacy of cancer treatment. Other features and advantages of the disclosure will be apparent from the detailed description, and from the claims.

### BRIEF DESCRIPTION OF THE DRAWINGS

FIG. 1 is a graph showing the relative ΔI_{M}/ΔI_{CD63} for MLH1, MSH2, MSH6, or PMS2 expression in extracellular vesicles present in plasma samples from healthy control and cancer patients.

FIG. 2 is a graph showing the relative ΔI_{M}/οI_{CD63} of extracellular vesicles present in normal and bladder cancer urine samples and comprising the MLH1, MSH6, and PMS2 markers.

FIGS. 3A and 3B illustrate MLH1, MSH2, MSH6, and PMS2 protein expression in HCT116 and SW480 cells. FIG. 3A is an image of a Western blot of MLH1, MSH2, MSH6, or PMS2 protein expression in HCT116 and SW480 cells. FIG. 3B is a heat map of MLH1, MSH2, MSH6, and PMS2 expression measured by iMES with extracellular vesicles derived from HCT116 and SW480 cells.

FIGS. 4A-4C illustrate the relative ΔI_{M}/ΔI_{CD63} for PD-1, PD-L1, and CD47 expression in extracellular vesicles present in plasma samples from healthy individuals and cancer patients.

FIG. 4A is a graph showing the relative ΔI_{M}/ΔI_{CD63} for the PD-L1 marker in extracellular vesicles present in plasma samples obtained from healthy individuals and from patients with gastric, lung, and colon cancer. FIG. 4B is a graph showing the relative ΔI_{M}/οI_{CD63} for the PD-1 marker in extracellular vesicles present in plasma samples obtained from healthy individuals and from patients with gastric, lung, and colon cancer. FIG. 4C is a graph showing the relative ΔI_{M}/οI_{CD63} for the CD47 marker in extracellular vesicles present in plasma samples obtained from healthy individuals and from patients with gastric, lung, and colon cancer.

FIGS. 5A and 5B illustrate the relative ΔI_{M}/οI_{CD63} for PD-L1 and CD47 expression in extracellular vesicles present in urine samples from healthy individuals and cancer patients. FIG. 5A is a graph showing the relative ΔI_{M}/οI_{CD63} for the PD-L1 marker in extracellular vesicles present in urine samples from healthy individuals and from individuals with bladder or prostate cancer. FIG. 5B is a graph showing the relative ΔI_{M}/οI_{CD63} for the CD47 marker in extracellular vesicles present in urine samples from healthy individuals and individuals with bladder or prostate cancer.

FIG. 6 is a graph showing the relative ΔI_{M}/οI_{CD63} for IL-12, perforin, and granzyme A expression in extracellular vesicles present in plasma samples from healthy individuals and from individuals with lung cancer.

FIG. 7 is a graph showing the relative ΔI_{M} for TCF7 expression in extracellular vesicles present in urine samples from healthy individuals and from individuals with bladder cancer.

FIG. 8 is a graph showing the relative ΔI_{M}/οI_{CD63} for Lin28A and Lin28B expression in extracellular vesicles present in plasma samples from healthy individuals and from individuals with gastric, lung, or colon cancer.

FIG. 9 is a graph showing the relative ΔI_{M}/οI_{CD63} for Lin28A and Lin28B expression in extracellular vesicles present in urine samples from healthy individuals and from individuals with bladder or prostate cancer.

### DETAILED DESCRIPTION

The disclosure features compositions and methods that are useful for predicting the efficacy of cancer treatment (e.g., a checkpoint inhibitor immunotherapy) and, in some embodiments, diagnosing or prognosing cancer. Also described herein are methods for detecting and characterizing cancer-associated markers in extracellular vesicles.

### Definitions

Unless defined otherwise, all technical and scientific terms used herein have the same meaning as commonly understood by those of ordinary skill in the art to which this disclosure pertains. The following references provide one of skill with a general definition of many of the terms used in this disclosure: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The phrase “immune checkpoint inhibitor” refers to the class of drugs that blocks certain proteins made by immune system cells (e.g., T cells) or by cancer cells. Immune checkpoint inhibitors are immunotherapy agents.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, the term “Covid-19” refers to an infectious disease caused by a novel coronavirus now called severe acute respiratory syndrome coronavirus 2 (SARS-CoV-2; formerly called 2019-nCoV).

By “marker” is meant any protein or polynucleotide having an alteration in expression level or activity that is associated with a disease or disorder.

By “MutL Homolog 1 (MLH1)” is meant a protein having at least 85% identity to NCBI Reference Sequence No. AAH06850.1 or a fragment thereof.

By “MutS Homolog 2 (MSH2)” is meant a protein having at least 85% identity to UniProt Reference Sequence No. P43246.1 or a fragment thereof.

By “MutS Homolog 6 (MSH6)” is meant a protein having at least 85% identity to NCBI Reference Sequence No. AAB39212.2 or a fragment thereof.

By “PMS1 Homolog 2 (PMS2)” is meant a protein having at least 85% identity to NCBI Reference Sequence No. NP 000526.2 or a fragment thereof.

By “Programmed Death Ligand 1 (PD-L1)” is meant a protein having at least 85% identity to NCBI Reference Sequence No. AAI13735.1 or a fragment thereof.

By “CD47” is meant a protein having at least 85% identity to NCBI Reference Sequence No. CAA49196.1 or a fragment thereof.

By “Programmed Cell Death 1 (PD-1)” is meant a protein having at least 85% identity to NCBI Reference Sequence No. AJS10360.1 or a fragment thereof.

By “perforin (PRF1)” is meant a protein having at least 85% identity to NCBI Reference Sequence No. BAG35621.1 or a fragment thereof.

By “granzyme A (GZMA)” is meant a protein having at least 85% identity to NCBI Reference Sequence No. CAG33249.1 or a fragment thereof.

By “Interleukin 2 (IL-2)” is meant a protein having at least 85% identity to NCBI

Reference Sequence No. AAH70338.1 or a fragment thereof.

By “Interleukin 6 (IL-6)” is meant a protein having at least 85% identity to NCBI Reference Sequence No. NP 000591.1 or a fragment thereof.

By “Interleukin 12 (IL-12)” is meant a protein having at least 85% identity to NCBI Reference Sequence No. NP_002178.2 or a fragment thereof.

By “Lin-28 Homolog A (LIN28A)” is meant a protein having at least 85% identity to NCBI Reference Sequence No. XP_011540450.1 or a fragment thereof.

By “Lin-28 Homolog B (LIN28B)” is meant a protein having at least 85% identity to NCBI Reference Sequence No. AAI37528.1 or a fragment thereof.

By “TCF7” is meant a protein having at least 85% identity to NCBI Reference Sequence No. NP_003193.2 or a fragment thereof.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

Any compositions or methods provided herein can be combined with one or more of any of the other compositions and methods provided herein.

The therapeutic methods described herein, in general, comprise administering a therapeutically effective amount of a pharmaceutical composition, such as a checkpoint inhibitor or other immunotherapy, to a subject in need thereof, including a mammal, particularly a human. Such treatment will be suitably administered to subjects, particularly humans, suffering from, having, susceptible to, or at risk of a disease, disorder, or symptom thereof. Determination of those subjects “at risk” can be made by any objective or subjective determination by a diagnostic test (e.g., genetic test, enzyme or protein marker, family history, and the like) or opinion of a subject or health care provider.

### Extracellular Vesicles (EVs)

Extracellular vesicles are membrane-bound phospholipid vesicles that are produced and secreted or otherwise released by cells, such as cells in healthy and unhealthy (e.g., cancerous) tissues. In some embodiments, the extracellular vesicles are exosomes. Extracellular vesicles can comprise cellular constituents (e.g., proteins, nucleic acids, and the like) from their originating cells, which can be used diagnostically. Combined with their relatively large abundance and ubiquitous presence in bodily fluids (e.g., urine, blood, ascites, CSF), extracellular vesicles offer significant advantages for cancer diagnosis, treatment, and management. Namely, extracellular vesicles represent “snapshots” of the molecular make-up of parent tumor cells, thus allowing an unbiased readout of the entire tumor. Furthermore, extracellular vesicles are abundant, relatively stable, and can be sampled repeatedly, thereby allowing monitoring of disease progression or response to treatment. The amount of extracellular vesicles and their molecular profiles have also been shown to correlate with tumor burden as well as treatment efficacy.

In some embodiments of the present disclosure, extracellular vesicles are derived from or present in samples obtained from a subject (e.g., a mammal, such as a human). A subject may not have or be suspected of having a disease or condition. In some embodiments, a subject has or is suspected of having a disease or condition, such as cancer, including but not limited to urothelial carcinoma, colorectal cancer, gastric cancer, lung cancer, renal cell carcinoma, head and neck cancer, melanoma, liver cancer, esophageal cancer, acute myeloid leukemia (AML), chronic myeloid leukemia (CML), glioblastoma, medulloblastoma, oral squamous cell carcinoma, breast cancer, ovarian cancer, cervical cancer, prostate cancer, testicular germ cell tumor, and Wilms tumor.

### Markers

As described herein, one or more markers in an extracellular vesicle may be used in an analysis to predict if a patient will respond favorably to therapy. In some embodiments, the therapy is a cancer therapy, such as an immunotherapy. In some embodiments, the therapy is a checkpoint inhibitor immunotherapy. Predictive makers contemplated in the present disclosure include, but are not limited to: i) mismatch repair proteins, ii) checkpoint proteins, iii) immune cell proteins, and iv) tumor associated proteins.

Mismatch repair proteins contemplated for analysis by the methods and compositions described herein include, but are not limited to, MLH1, MSH2, MSH6, and PMS2.

Checkpoint proteins contemplated for analysis by the methods and compositions described herein include, but are not limited to, PD-1, PD-L1, CD80 (B7-1), CD86 (B7-2), and CD47. These checkpoint proteins are expressed in T cells and in some types of cancer cells. Those cancers expressing checkpoint inhibitors can be susceptible to immune checkpoint inhibitor therapy, or checkpoint protein inhibitor immunotherapy, which comprises therapeutic agents and drugs that inhibit the function certain checkpoint proteins. When these checkpoint proteins are inhibited or blocked, the “brakes” on the immune system are released and T cells are more effective at killing cancer cells.

Immune cell proteins contemplated for analysis by the methods and compositions presently described include, but are not limited to, perforin, granzymes (e.g., granzyme A), IL-2, IL-6, IL-12 and TCF7.

Tumor associated proteins (TAPs) contemplated herein for analysis with the presently described methods and compositions include, but are not limited to, LIN28A and LIN28B. LIN28 is an RNA binding protein with two isoforms (LIN28A and LIN28B) that share similar structure and function. LIN28A is predominantly cytoplasmic and has been detected in association with ribosomes, P-bodies, and stress granules. Subcellular localization of LIN28B is less clear. It may shuttle between the cytoplasm and the nucleus in a cell-cycle dependent manner. Activation of either LIN28A or LIN28B results in post-transcriptional down-regulation of the let-7 microRNA family observed in many cancers. The decrease in let-7 microRNAs leads to overexpression of their targets such as MYC, RAS, HMGA2, BLIMP1, as well as PD-L1. Upregulation of LIN28 correlates with advanced disease and poor prognosis. Thus, in some embodiments, Lin28 provides diagnostic and predictive value for disease detection as well as for treatment prediction, an advance over the prior art because LIN28-based liquid biopsy has not been reported to date. For example, in some embodiments, the presence of LIN28 in extracellular vesicles is used to detect cancer in patients using the methods described herein. In some embodiments, the cancer that is detected by analyzing the presence of LIN28 in extracellular vesicles includes, but is not limited to, urothelial carcinoma, colorectal cancer, gastric cancer, lung cancer, renal cell carcinoma, head and neck cancer, melanoma, liver cancer, esophageal cancer, acute myeloid leukemia (AML), chronic myeloid leukemia (CML), glioblastoma, medulloblastoma, oral squamous cell carcinoma, breast cancer, ovarian cancer, cervical cancer, prostate cancer, testicular germ cell tumor, and Wilms tumor.

In certain embodiments, a combination of markers can be used, such as any of the following listings:

### 1) MLH1, MSH2, PD-L1, CD47, and Lin28;

### 2) MLH1, MSH2, PD-L1, CD47, and IL-12;

### 3) MLH1, MSH2, PD-L1, CD47, and TCF7;

### 4) MLH1, MSH2, PD-L1, TCF7, and Lin28;

### 5) MLH1, MSH2, PD-L1, IL-12, and Lin28;

### 6) MLH1, MSH2, PD-L1, IL-12 and TCF7;

### 7) MLH1, MSH2, IL-12, TCF7 and Lin28;

### 8) PD-L1, CD47, IL-12, TCF7, and Lin28;

### 9) MLH1, MSH2, PD-L1, CD47, TCF7, and Lin28;

### 10) MLH1, MSH2, PD-L1, CD47, TCF7, and IL-12;

### 11) MLH1, MSH2, PD-L1, CD47, TCF7, IL-12, and Lin28;

### 12) MLH1, MSH2, PD-L1, Perforin, Granzymes, IL-12, and TCF7

### 13) MLH1, MSH2, PD-L1, Perforin, Granzymes, IL-12, TCF7, and Lin28;

### 14) MLH1, MSH2, PD-L1, CD47, Granzymes, IL-12, TCF7, and Lin28;

### 15) MLH1, MSH2, PD-L1, CD47, Perforin, IL-12, TCF7, and Lin28;

### 16) MLH1, MSH2, PD-L1, CD47, Perforin, Granzymes, TCF7, and Lin28;

### 17) MLH1, MSH2, PD-L1, CD47, Perforin, Granzymes, IL-12, TCF7, and Lin28; or

### 18) MLH1, MSH2, MSH6, PMS2, PD-L1, CD47, PD-1, Perforin, Granzymes, IL-2, IL-6, IL-12, TCF7, and Lin28.

iMES

Addressing the unmet clinical need for extracellular vesicle analysis, integrated Magneto-Electronic Sensing (iMES) technology was recently developed. iMES technology combines extracellular vesicle isolation and detection in a single platform and offers distinct advantages: i) tumor-specific extracellular vesicles can be analyzed directly from complex media without the need for extensive filtration or centrifugation; ii) the assay can achieve high detection sensitivity through magnetic enrichment and signal amplification; and iii) based on the electronic detection scheme, sensors can be miniaturized and expanded for parallel measurements. The first-generation iMES prototype outperformed conventional assays in several aspects. For example, detection sensitivity of the first-generation iMES prototype was >1000-fold higher compared to enzyme-linked immunosorbent assay (ELISA), and the iMES assay was fast (<1 hour total assay time) and affordable. Furthermore, iMES assays only consumed ˜10 μl of plasma samples. In pilot clinical studies, iMES successfully detected cancer extracellular vesicles in patient plasma and urine samples.

The iMES technology is used to measure the expression level of specific extracellular forms of proteins (markers) in the biofluids. In some embodiments, an analyte detection device is used to detect markers in an extracellular vesicle. In some embodiments, the analyte detection device is an integrated magnetic-electrochemical sensing (iMES) system or device, such as those described in International Application No. PCT/US2017/015433, the contents of which are herein incorporated by reference in their entirety. Such a system or device can include a miniaturized magnetic electrochemical sensing system that detects extracellular vesicles in a sample (e.g., a biological sample). In some embodiments, the iMES technology described herein can be used to determine the iMES metrics of the markers in the sample.

Both single marker iMES metric(s) and an integrated score can be used to predict a patient's responsiveness to therapy or to a particular therapeutic agent or combination of therapeutic agents. In some embodiments, an integrated score, when compared to a control (e.g., healthy subject), in statistically indicative of the effectiveness of a therapeutic modality (e.g., cancer immunotherapy). In certain embodiments, the integrated score itself can be used as a diagnostic or prognostic modality. It should be noted, however, that while the integrated score can reflect a difference than the control, the individual biological level (e.g., amount of EV-associated protein) of each specific marker can be increased, decreased or unchanged compared to the control.

Exemplary Scoring Methodologies Include:

### 1. SingleMarkerScore Method

By “SingleMarkerScore” is meant the iMES metric of a specific predictive marker. The SingleMarkerScore above or below a set threshold value can be used to predict if a patient is more likely to respond to checkpoint inhibitor immunotherapy. In some embodiments, the threshold value for each marker is determined using retrospective patient responsiveness data. In some embodiments, the threshold value for each marker is a reference value known to those skilled in the art. In some embodiments, a SingleMarkerScore is deemed predictive of a specific marker when the score is greater or lesser than the threshold value by a predetermined amount (e.g., a set percentage or fraction of the threshold value, for example 10% or 200%, one or more standard deviations, and the like).

### 2. CategoryScore Method

As used herein “CategoryScore” refers to the total number of effective categories of markers for which a patient has an iMES metric that deviates from the threshold value to such an extent that the iMES metric is deemed significant, which indicates that the category is “effective.” The greater the number of effective categories, the higher the score assigned to the patient by the CategoryScore method. In some embodiments, the threshold values in each category are set using retrospective patient responsiveness data. In some embodiments, the threshold value for each marker is a reference value known to those skilled in the art. A patient's CategoryScore could have a value of 0, 1, 2, 3, or 4. The higher a CategoryScore, the stronger the prediction that the patient is more likely to respond to checkpoint inhibitor immunotherapy. The categories included in the CategoryScore method include the mismatch repair (MMR) category, the checkpoint protein category, the immune marker category, and the tumor-associated protein category.

The mismatch repair category comprises any marker or protein associated with the mismatch repair process in eukaryotic cells. Examples of markers associated with mismatch repair include, but are not limited to, MLH1 and MSH2. Thus, if MLH1 or MSH2 level is below their respective set values, the MMR category is considered an effective category to be included in the overall CategoryScore.

The checkpoint protein category comprises any marker or protein associated with a checkpoint function. Examples of marker or proteins associated with a checkpoint function include, but are not limited to, PD-1, PD-L1, and CD47. For example, if the iMES metric for PD-1 or PD-L1 is above its set threshold value, the checkpoint category is considered effective for PD-1/PD-L1 inhibitors and is included in the overall CategoryScore. If the iMES metric for CD47 is above a set threshold value, the checkpoint category is considered effective for CD47 inhibitors and is included in the overall CategoryScore.

The immune marker category comprises one or more markers associated with immune response to a cancer cell or tumor. Examples of immune markers include, but are not limited to perforin, granzyme A, IL-2, IL-12, and TCF7. If one of the immune markers (perforin, granzymes, IL-2, IL-12, or TCF7) is above their respective set values, the immune marker category is considered an effective category to be included in the overall CategoryScore calculation.

The tumor-associated protein (TAP) category comprises one or more markers associated with a tumor. Examples of TAP markers includes, but is not limited to, LIN28A and LIN28B. If the iMES metric for LIN28A or LIN28B is above its set threshold value, the TAP category is considered an effective category to be included in the overall CategoryScore calculation.

### 3. RegressionScore Method

As used herein, “RegressionScore” refers to the weighted sum of the adjusted iMES metric of all the tested markers. For example, in the case of MMR markers, the adjusted iMES metric for each MMR marker tested is the reciprocal of the original iMES metric for each MMR marker. If the RegressionScore (i.e., the weighted sum of the adjusted iMES metrics for each marker tested) is above (or below) the set threshold value, a patient is more likely to respond to checkpoint inhibitor immunotherapy. In some embodiments, the weights and the threshold value are determined using retrospective patient responsiveness data. In some embodiments, the weights or the threshold value are determined using retrospective patient responsiveness data. In some embodiments, the one or more of the weights used in determining the Regression score is a reference value known to those skilled in the art. In some embodiments, the threshold value for each marker is a reference value known to those skilled in the art. In some embodiments, the adjusted iMES metric is the logarithm of the original iMES metric. In some embodiments, the adjusted iMES metric is the reciprocal of the original iMES metric.

### 4. DecisionTreeScore Method

By “DecisionTreeScore” is meant the output of a decision tree model using the iMES metric of all the tested markers as input. In some embodiments, the parameters of the decision tree model used in generating the DecisionTreeScore are determined using retrospective patient responsiveness data. In some embodiments, the parameters of the decision tree model used in generating the DecisionTreeScore are determined using parameters known to those skilled in the art. In some embodiments, the DecisionTreeScore can be “yes” or “no,” or any other nomenclature that indicates affirmation or negation. In some embodiments, the DecisionTreeScore can indicate that the results of the analysis are inconclusive (i.e., the results do not support an affirmative or a negative output). If the DecisionTreeScore output is affirmative, the results indicate that a patient is more likely to respond to a cancer therapy (e.g., checkpoint inhibitor immunotherapy) than a patient whose DecisionTreeScore is negative.

### 5. NeuralNetworkScore Method

The term “NeuralNetworkScore” is defined as the output of a multi-layer feed forward neural network model using the iMES metric of all the tested markers as input. The parameters of the neural network model and threshold values are determined using retrospective patient responsiveness data. In some embodiments, the parameters used in generating the NeuralNetworkScore are determined using algorithms known to those skilled in the art. In some embodiments, the neural network model has one hidden layer. In some embodiments, the neural network model has multiple hidden layers. In some embodiments, the threshold values against which the NeuralNetworkScore is compared are values known to those skilled in the art. If the NeuralNetworkScore is above the threshold, a patient is more likely to respond to a cancer therapy (e.g., checkpoint inhibitor immunotherapy).

Mismatch Repair (MMR) Protein Detection Using iMES

In some embodiments, the presence of mismatch repair (MMR) proteins in biofluids can be detected using the iMES technology.

For instance, MMR proteins can be released to biofluids and associated with extracellular vesicles (EVs). Magnetic beads conjugated to antibodies specific to EV markers can capture EVs from a biological sample (e.g., urine, plasma, conditioned cell medium). In turn, the sample can be treated with secondary molecules specific to MMR (e.g., a labeling ligand, such as biotinylated antibodies specific against MMR proteins), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the MMR expressing EVs have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of MMR expressing EVs. In turn, this current can be measured by an MCU or other computing device, and the resulting information can be used for investigative or predictive purposes. For example, a relatively low current can correspond to a relatively low concentration of MMR expressing EVs, and may be an indicator of mismatch repair deficiency or microsatellite instability in a patient.

In some cases, the output of a potentiostat can be compared to a threshold or reference level, and the presence or absence of a mismatch repair protein in a patient can be detected based on the comparison. For example, if the output of a potentiostat or the combined output of several potentiostats are sufficiently high (e.g., a current that exceeds a reference or threshold level), a prediction that the patient is less likely to respond to checkpoint inhibitor immunotherapy may be rendered. However, if the output of the potentiostat or the combined output of several potentiostats are relatively low (e.g., a current that does not exceed the reference or threshold level), a prediction that the patient is more likely to respond to checkpoint inhibitor immunotherapy may be rendered. In some cases, an instrument can render analyses automatically or semi-automatically based on the measurements.

Although the detection of MMR in blood, urine, or conditioned medium is described above, this is merely an example. In practice, MMR proteins can be either free-floating (e.g., free-floating in plasma, urine, or any other biological sample) or expressed on the surface or inside of a biological structure (e.g., extracellular vesicle).

Checkpoint Protein Detection Using iMES

In some embodiments, the presence of checkpoint proteins (PD-1, PD-L1, CD47) in biofluids can be detected using the iMES technology.

For instance, checkpoint proteins can be released to biofluids and associated with extracellular vesicles (EVs). Magnetic beads conjugated to antibodies specific to EV markers can capture EVs from a biological sample (e.g., urine, plasma, conditioned cell medium). In turn, the sample can be treated with secondary molecules specific to checkpoint proteins (e.g., a labeling ligand, such as biotinylated antibodies specific against checkpoint proteins), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the checkpoint protein expressing EVs have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of checkpoint protein expressing EVs.

In turn, this current can be measured by an MCU or other computing device, and the resulting information can be used for investigative, diagnostic or predictive purposes. For example, a relatively high current can correspond to a relatively high concentration of checkpoint proteins, and may be an indicator of cancer in a patient, or a predictor that the patient is more likely to respond to checkpoint inhibitor immunotherapy.

In some cases, the output of a potentiostat can be compared to a threshold or reference level, and the presence or absence of mismatch repair deficiency in a patient can be detected based on the comparison. For example, if the output of a potentiostat is sufficiently high (e.g., a current that exceeds a reference or threshold level), a prediction that that the patient is more likely to respond to checkpoint inhibitor immunotherapy can be rendered. However, if the output of the potentiostat is relatively low (e.g., a current that does not exceed the reference or threshold level), a prediction that the patient is less likely to respond to checkpoint inhibitor immunotherapy may be rendered. In some cases, an instrument can render analyses automatically or semi-automatically based on the measurements.

Although the detection of checkpoint proteins in blood or urine is described above, this is merely an example. In practice, checkpoint proteins can be either free-floating (e.g., free-floating in plasma, urine, or any other biological sample) or expressed on the surface or inside of a biological structure (e.g., extracellular vesicle).

Immune Markers Detection Using iMES

In some embodiments, the presence of immune proteins (IL-2, IL-6, IL-12, Perforin, Granzyme, TCF7) in biofluids can be detected using the iMES technology.

For instance, immune proteins can be released to biofluids and associated with extracellular vesicles (EVs). Magnetic beads conjugated to antibodies specific to EV markers can capture EVs from a biological sample (e.g., urine, plasma, conditioned cell medium). In turn, the sample can be treated with secondary molecules specific to immune proteins (e.g., a labeling ligand, such as biotinylated antibodies specific against immune proteins), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the immune protein expressing EVs have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of immune protein expressing EVs. In turn, this current can be measured by an MCU or other computing device, and the resulting information can be used for investigative, or predictive purposes. For example, a relatively high current can correspond to a relatively high concentration of specific immune proteins, and may be a predictor that the patient is more likely or less likely to respond to checkpoint inhibitor immunotherapy.

In some cases, the output of a potentiostat can be compared to a threshold or reference level, and the presence or absence of mismatch repair deficiency in a patient can be detected based on the comparison. For example, if the output of a potentiostat is sufficiently high (e.g., a current that exceeds a reference or threshold level), a prediction that that the patient is more likely or less likely to respond to checkpoint inhibitor immunotherapy can be rendered. In some cases, an instrument can render analyses automatically or semi-automatically based on the measurements.

Although the detection of immune proteins in blood is described above, this is merely an example. In practice, immune proteins can be either free-floating (e.g., free-floating in plasma, urine, or any other biological sample) or expressed on the surface or inside of a biological structure (e.g., extracellular vesicle).

Lin28 Detection Using iMES

In some embodiments, the presence of Lin28 proteins in biofluids can be detected using the iMES technology.

For instance, Lin28 proteins can be released to biofluids and associated with extracellular vesicles (EVs). Magnetic beads conjugated to antibodies specific to EV markers can capture EVs from a biological sample (e.g., urine, plasma, conditioned cell medium). In turn, the sample can be treated with secondary molecules specific to Lin28 (e.g., a labeling ligand, such as biotinylated antibodies specific against checkpoint proteins), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the Lin28 expressing EVs have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of Lin28 expressing EVs. In turn, this current can be measured by an MCU or other computing device, and the resulting information can be used for investigative, diagnostic or predictive purposes. For example, a relatively high current can correspond to a relatively high concentration of Lin28 proteins, and may be an indicator of cancer in a patient.

In some cases, the output of a potentiostat can be compared to a threshold or reference level, and the presence or absence of cancer in a patient can be detected based on the comparison. For example, if the output of a potentiostat is sufficiently high (e.g., a current that exceeds a reference or threshold level), a diagnosis regarding the presence of a cancer can be rendered. However, if the output of the potentiostat is relatively low (e.g., a current that does not exceed the reference or threshold level), a diagnosis regarding the absence of a cancer may be rendered. In some cases, an instrument can render analyses automatically or semi-automatically based on the measurements.

Although the detection of Lin28 in blood or urine is described above, this is merely an example. In practice, checkpoint proteins can be either free-floating (e.g., free-floating in plasma, urine, or any other biological sample) or expressed on the surface or inside of a biological structure (e.g., extracellular vesicle).

### Methods and Uses

Methods are provided herein for predicting the efficacy of a cancer therapy. In some embodiments, the methods comprise detecting and characterizing one or more markers associated with a disease (e.g., cancer) in a sample. In some embodiments, the sample is obtained from a subject (e.g., a mammal, such as a human). In some embodiments, the sample comprises a liquid biopsy. In some embodiments, the sample is any biological sample. In some embodiments the sample is a bodily fluid including, but not limited to, blood, serum, plasma, saliva, gastric acid, cerebrospinal fluid, synovial fluid, lymph, aqueous humor, or urine.

In some embodiments, detecting the one or more markers in an extracellular vesicle comprises an immunoassay (e.g., antibody-mediated sequestration or labeling of the one or more markers in the extracellular vesicle). In some embodiments, an iMES or analogous system or device is employed to detect the markers present in extracellular vesicles. A device analogous to an iMES system or device will enable the capture or detection of extracellular vesicles and/or the markers present in, on, or associated with extracellular vesicles in a sample. For example, extracellular vesicles may be captured using an affinity capture agent capable of binding or otherwise interacting with the membrane of an extracellular vesicle. In some embodiments, the extracellular vesicle is effectively captured by an antibody that specifically binds to a marker present in or on the extracellular vesicle. In some embodiments, the extracellular vesicle is permeabilized prior to marker labeling. In some embodiments, the extracellular vesicle is broken open prior to maker labeling.

For example, magnetic beads can be conjugated to antibodies that specifically bind one or more markers of interest. The magnetic beads are mixed with a biological sample containing extracellular vesicles (e.g., blood or urine), such that extracellular vesicles expressing the one or more markers are bound by the antibody conjugated to the bead. The bead can then be isolated from the sample with a magnet. In some embodiments, the sample is treated with a secondary molecule that specifically binds to a marker on the extracellular vesicle (e.g., a labeling ligand, such as biotinylated antibodies specific against the one or more markers). In some embodiments, the sample is treated with a tertiary composition comprising a moiety that specifically binds the secondary molecules. In some embodiments, the tertiary composition further comprises a moiety having oxidizing enzymatic activity (e.g., a streptavidin-HRP). The sample can then be combined with an electron mediator solution (e.g., a solution containing 3,3′,5,5′-tetramethylbenzidine, TMB). The voltage or current generated can vary due to the oxidation-reduction reaction. The currents generated are detected using a sensing system, such as those described herein, and especially at Example 10, and the collected data can be used for predictive or diagnostic purposes. As the assay described herein is minimally invasive, a patient's disease can be monitored with minimal risk to the patient and in a cost-effective manner.

The data generated can be analyzed to determine whether a patient's cancer will be responsive to a particular therapy. For example, the data can be analyzed to determine the amount of a marker or markers present in extracellular vesicles in the sample, and this amount can be compared to a reference amount. Deviations from the reference amount indicate, in some embodiments, that a subject's cancer will be responsive to a particular therapy (e.g., a checkpoint inhibitor immunotherapy). Additionally, data for all markers can be scored using any of the methods described herein.

In some aspects, a patient whose cancer has been predicted to be effectively treated by a particular therapy is then administered the therapy. Administration of the therapies contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

The present disclosure also contemplates monitoring the treatment or prognosis of a subject having cancer. Using the methods and compositions described herein, samples obtained from the subject can be tested at different timepoints during the course of treatment. In some embodiments, a first sample is obtained from the patient prior to commencing treatment. In some embodiments, subsequent samples are obtained after treatment has begun. By analyzing extracellular vesicles for markers associated with cancer, determinations can be made as to whether the treatment is effective. For example, in some cases, a reduced amount of a marker after treatment is indicative of improvement in the patient's condition. Conversely, if the amount of markers observed remains static, the therapy is not effectively treating disease. Additionally, if additional markers that were not present in the initial sample appear during the course of treatment, the patient's cancer may be progressing.

### Kits

One aspect of the present disclosure provides a kit for the detection and characterization of markers present in an extracellular vesicle. In some embodiments, the kit comprises magnetic beads conjugated to a capture molecule (e.g., an antibody) that specifically binds one or more of the markers described herein or another marker associated with cancer. In some embodiments, the kit comprises a secondary molecule that specifically binds to a ligand on a extracellular vesicle (e.g., a labeling ligand, such as biotinylated antibodies specific against the one or more markers). In some embodiments, the kit further comprises a tertiary composition comprising a moiety that specifically binds the secondary molecules. In some embodiments, the tertiary composition further comprises a moiety having oxidizing enzymatic activity (e.g., a streptavidin-HRP). In some embodiments, the kit comprises an electron mediator solution (e.g., a solution containing 3,3′,5,5′-tetramethylbenzidine, TMB).

In some embodiments, the kit comprises a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding reagents.

The kit comprises a substrate comprising a plurality of electrodes on a first surface of the substrate, wherein a set of electrodes of the plurality of electrodes defines a sample detection region. In some embodiments, the electrodes are screen printed electrodes. In some embodiments, the electrodes are printed circuit board electrodes.

The kit may provide instructions for using the kit to detect the markers of interest. The instructions will generally include information about the use of the kit for the detection and characterization of markers associated with cancer (e.g., one or more of the markers described herein). In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization.

In certain embodiments, a therapeutic approach can be predicted to be effective or ineffective in treating a subject's cancer by applying a biological sample (e.g., blood, plasma, or urine) from the subject to the kit, or components thereof, and detecting and characterizing (e.g., determining amounts of) the relevant markers that are specifically bound by capture molecules. By way of example, the method comprises: (i) obtaining a sample from the subject; (ii) adding subject's sample to the components in the kit, e.g., a holding tube or a substrate; and (iii) detecting and characterizing the capture molecules to which the markers in the sample have bound.

The following examples are put forth so as to provide those of ordinary skill in the art with a complete disclosure and description of how to make and use the assay, screening, and therapeutic methods of the disclosure, and are not intended to limit the scope of the disclosure.

### EXAMPLES

### Example 1. Direct Plasma Analyses of MMR from Patients with Gastric, Lung, or Colon Cancer, and from Healthy Control

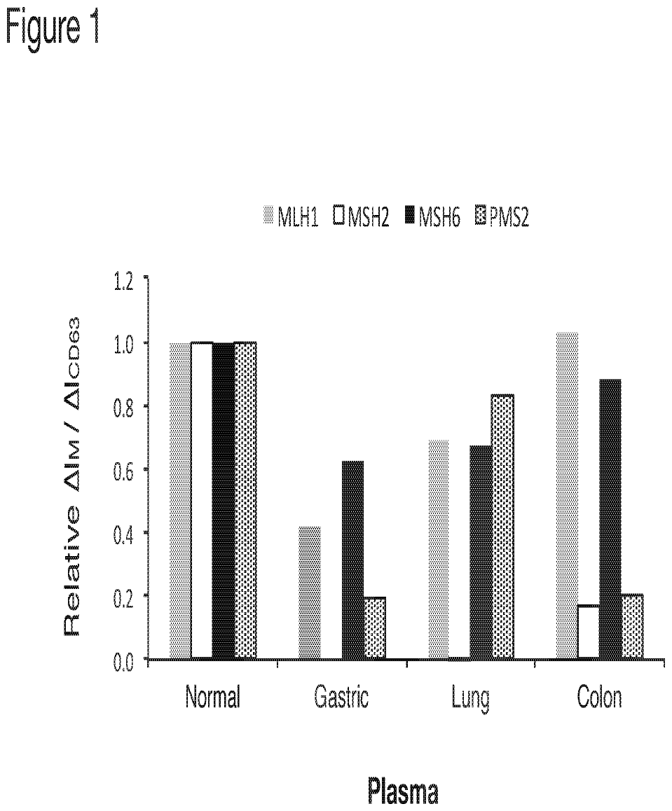

Clinical plasma samples were aliquoted without any purification, and each aliquot (10 μL, per marker) was incubated with magnetic beads for EV capture. The bead-bound EVs were consecutively labeled for target markers and HRP, and loaded onto the device. Five different markers (CD63, MLH1, MSH2, MSH6 and PMS2) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control plasma as 1, and the relative ΔI_{M}/ΔI_{CD63} metric from each cancer plasma was normalized and plotted (FIG. 1). MMR proteins (MLH1, MSH2, MSH6, PMS2) were present in EVs from pooled healthy control plasma. Specific MMR protein loss was detected in EVs from some cancer plasma samples.

### Example 2. Direct Urine Analyses of MMR from Patients with Bladder Cancer, and from Healthy Control

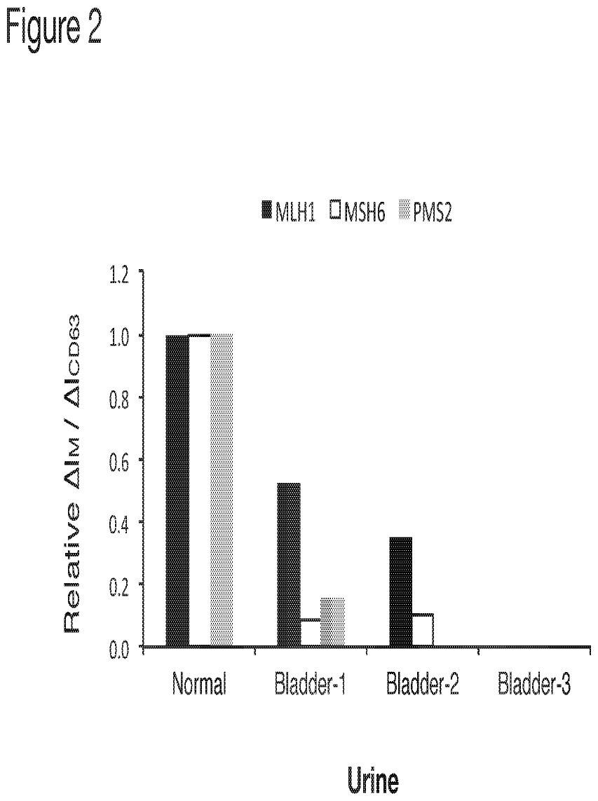

Clinical urine samples were aliquoted without any purification, and each aliquot (80 μL per marker) was incubated with magnetic beads for EV capture. The bead-bound EVs were consecutively labeled for target markers and HRP, and loaded onto the device. Four different markers (CD63, MLH1, MSH6 and PMS2) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control urine as 1, and the relative ΔI_{M}/ΔI_{CD63} metric from each bladder cancer urine was normalized and plotted (FIG. 2). MMR proteins (MLH1, MSH6, PMS2) were present in EVs from pooled healthy control urine. MMR protein loss was detected in EVs from bladder cancer urine samples.

### Example 3. Direct Conditioned Medium Analyses of MMR from HCT116 and SW480 Colorectal Cancer Cell Lines

Conditioned cell culture medium were aliquoted without any purification, and each aliquot (80 μL per marker) was incubated with magnetic beads for EV capture. The bead-bound

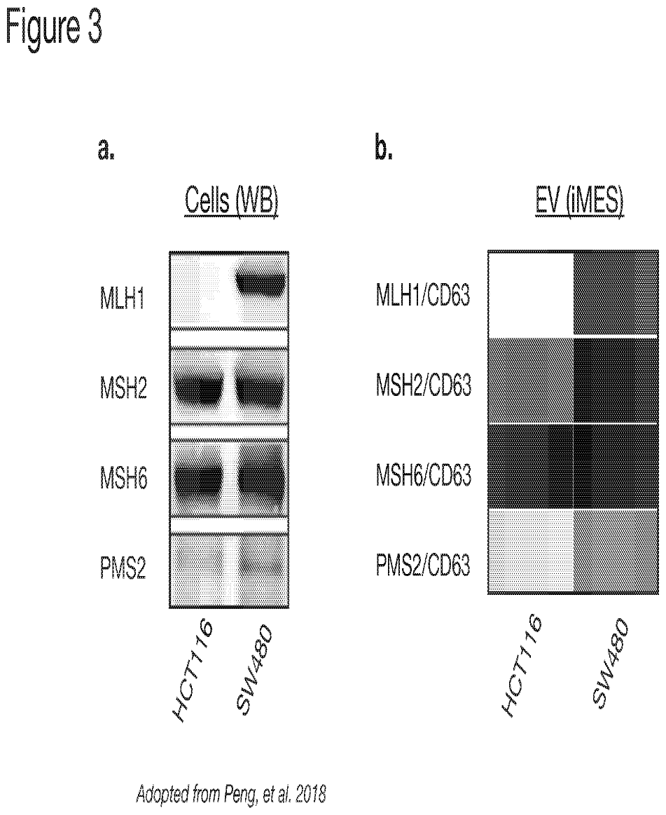

EVs were consecutively labeled for target markers and HRP, and loaded onto the device. Five different markers (CD63, MLH1, MSH2, MSH6 and PMS2) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). Heat map was generated based on ΔI_{M}/ΔI_{CD63} metric to show the relative EV marker expression across different cell lines. The molecular profiles of EVs and cells [adopted from Peng et al 2018 (DOI: 10.3892/or.2018.6510)] were highly correlated (FIGS. 3A and 3B).

### Example 4. Direct Plasma Analyses of Checkpoint Proteins from Patients with Gastric, Lung, or Colon Cancer, and from Healthy Control

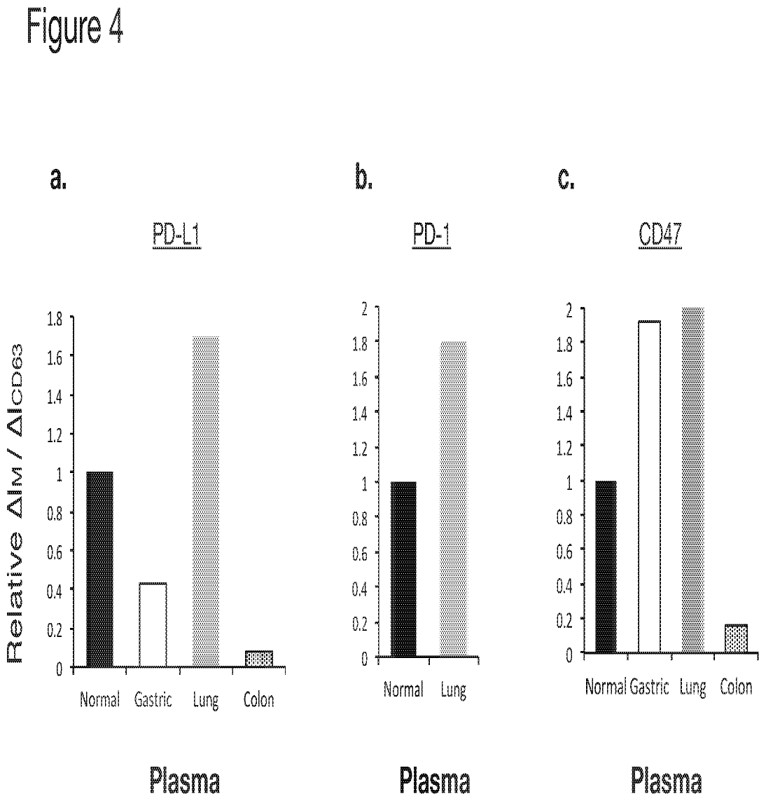

Clinical plasma samples were aliquoted without any purification, and each aliquot (10 μL, per marker) was incubated with magnetic beads for EV capture. The bead-bound EVs were consecutively labeled for target markers and HRP, and loaded onto the device. Four different markers (CD63, PD-1, PD-L1, CD47) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control plasma as 1, and the relative ΔI_{M}/ΔI_{CD63} metric from each cancer plasma was normalized and plotted (FIGS. 4A-4C). Checkpoint proteins (PD-L1, PD-1, CD47) were present in EVs from pooled healthy control plasma. Checkpoint protein overexpression was detected in EVs from some cancer plasma samples.

### Example 5. Direct Urine Analyses of Checkpoint Proteins from Patients with Bladder or Prostate Cancer, and from Healthy Control

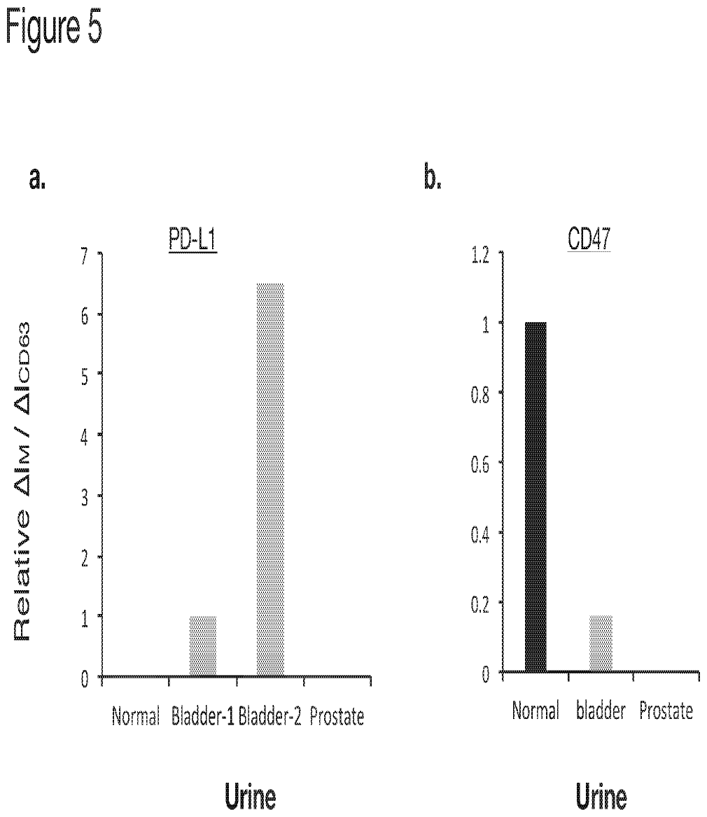

Clinical urine samples were aliquoted without any purification, and each aliquot (80 μL per marker) was incubated with magnetic beads for EV capture. The bead-bound EVs were consecutively labeled for target markers and HRP, and loaded onto the device. Four different markers (CD63, PD-1, PD-L1 and CD47) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from one bladder cancer urine as 1, and the relative ΔI_{M}/ΔI_{CD63} metric from healthy control and additional cancer urine samples were normalized and plotted (FIGS. 5A-5B). PD-L1 was not detected in EVs from pooled healthy control or prostate cancer urine samples, but present in EVs from bladder cancer urine samples. CD47 was not detected in EVs from prostate cancer urine samples, but present in EVs from healthy control and bladder cancer urine samples.

### Example 6. Direct Plasma Analyses of Immune Markers from Patients with Lung Cancer, and from Healthy Control

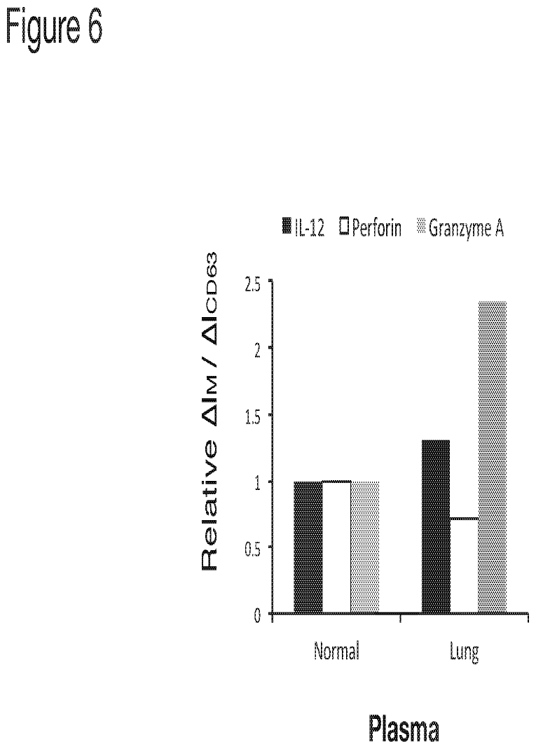

Clinical plasma samples were aliquoted without any purification, and each aliquot (10 μL per marker) was incubated with magnetic beads for EV capture. The bead-bound EVs were consecutively labeled for target markers and HRP, and loaded onto the device. Four different markers (CD63, IL-12, Perforin, and Granzyme A) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control plasma as 1, and the relative ΔI_{M}/ΔI_{CD63} metric from lung cancer plasma was normalized and plotted (FIG. 6). Immune markers (IL-12, Perforin, Granzyme A) were present in EVs from pooled healthy control plasma. Granzyme A overexpression was detected in EVs from the lung cancer plasma sample.

### Example 7. Direct Urine Analyses of Immune Markers from Patients with Bladder Cancer, and from Healthy Control

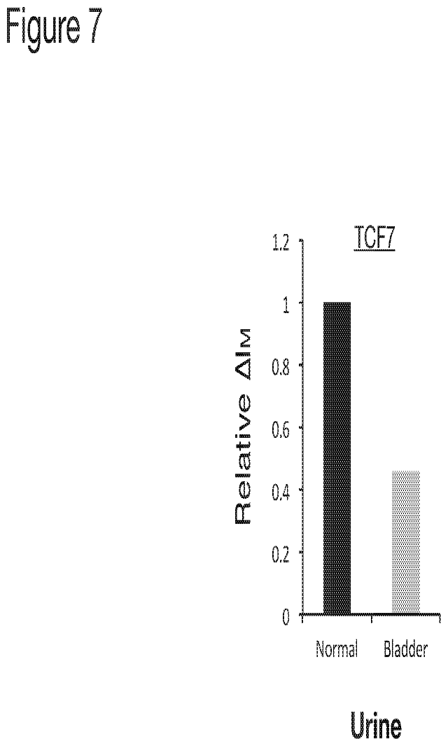

Clinical urine samples were aliquoted without any purification, and each aliquot (80 μL per marker) was incubated with magnetic beads for EV capture. The bead-bound EVs were consecutively labeled for target markers and HRP, and loaded onto the device. CD63 and TCF7 were measured with iMES along with their respective IgG-controls. The net signal difference ΔI_{M}(I_{CD63+M}−I_{IgG+M}; was obtained. We used ΔI_{CD63} to estimate the total EV load, and defined ΔI_{M} as the expression level of a target marker (M). We then set the ΔI_{M} metric from the pooled healthy control urine as 1, and the relative ΔI_{M} metric from bladder cancer urine was normalized and plotted (FIG. 7). TCF7 was present in EVs from pooled healthy control urine and bladder cancer urine.

### Example 8. Direct Plasma Analyses of Lin28 from Patients with Gastric, Lung, or Colon Cancer, and from Healthy Control

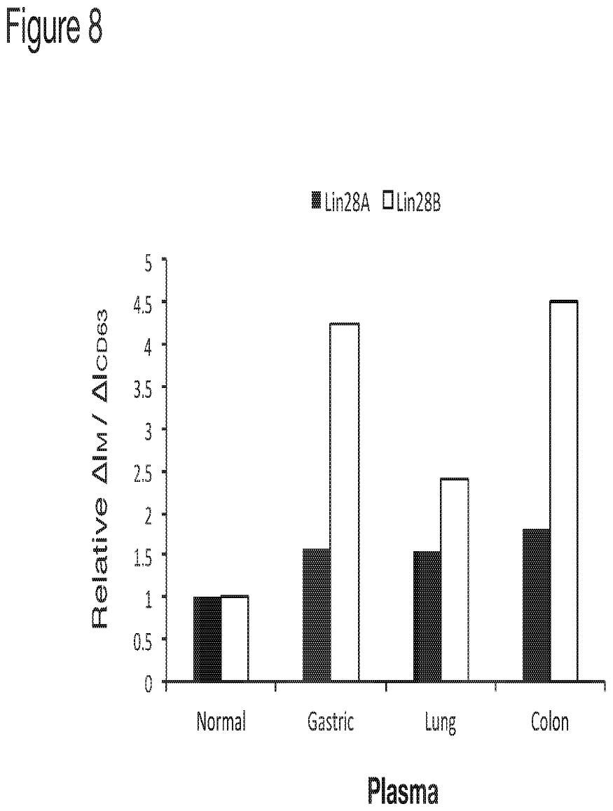

Clinical plasma samples were aliquoted without any purification, and each aliquot (10 μL per marker) was incubated with magnetic beads for EV capture. The bead-bound EVs were consecutively labeled for target markers and HRP, and loaded onto the device. Three different markers (CD63, Lin28A, and Lin28B) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control plasma as 1, and the relative ΔI_{M}/ΔI_{CD63} metric from each cancer plasma was normalized and plotted (FIG. 8). Lin28A and Lin28B were present in EVs from pooled healthy control plasma. Lin28A and/or Lin28B overexpression was detected in EVs from tested cancer plasma samples.

### Example 9. Direct Urine Analyses of Lin28 from Patients with Bladder or Prostate Cancer, and from Healthy Control

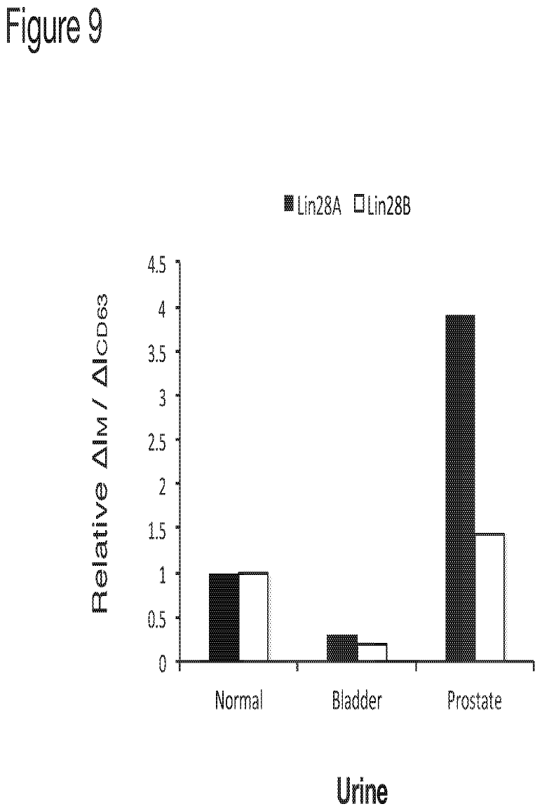

Clinical urine samples were aliquoted without any purification, and each aliquot (804 per marker) was incubated with magnetic beads for EV capture. The bead-bound EVs were consecutively labeled for target markers and HRP, and loaded onto the device. Three different markers (CD63, Lin28A, and Lin28B) were measured with iMES along with their respective IgG-controls. The net signal difference ΔI_{M}(I_{CD63+M}−I_{IgG+M}; was obtained. We used ΔI_{CD63} to estimate the total EV load, and defined a normalized metric ΔI_{M}/ΔI_{CD63} as the expression level of a target marker (M). We then set the ΔI_{M}/ΔI_{CD63} metric from the pooled healthy control urine as 1, and the relative ΔI_{M}/ΔI_{CD63} metric from each bladder cancer urine was normalized and plotted (FIG. 9). Lin28A and Lin28B were present in EVs from pooled healthy control urine. Lin28A overexpression was detected in EVs from tested prostate urine.

### Example 10. Direct Plasma Analyses of Immune Markers from Patients Presented with Both Cancer and Coronavirus Disease 2019 (COVID-19)

Studies suggest that COVID-19 in patients with cancer is associated with higher rates of hospitalization and severe outcomes, and treatment with immunotherapy (e.g. immune checkpoint inhibitors) may lead to even more server respiratory illness in some patients. There is an urgent need to identify and monitor the cancer patients who are likely to benefit from immunotherapy and also have a more favorable Covid-19 outcome.

Clinical plasma samples (10 μL per marker) can be incubated with magnetic beads for EV capture. The bead-bound EVs can be consecutively labeled for target markers and HRP, and loaded onto the device. Immune markers (CD63, IL-6, IL-12, Perforin) can be measured with iMES along with their respective IgG-controls. The net signal difference ΔI_{M}(I_{CD63+M}−I_{Ig+M}) can be obtained. ΔI_{CD63} can be used to estimate the total EV load, and define a normalized metric ΔI_{M}/ΔI_{CD63} as the expression level of a target marker (M). The ΔI_{M}/ΔI_{CD63} metric can then be set from the pooled healthy control plasma as 1, and the relative ΔI_{M}/ΔI_{CD63} metric from patient plasma can be normalized and plotted. The relative ΔI_{M}/ΔI_{CD63} metric of immune markers (IL-6, IL-12, Perforin) can be used as a prognostic modality for immunotherapy as well as for Covid-19 progression.

### Example 11: Materials and Methods

### Cell Lines and Clinical Samples

Colorectal cancer cell lines SW480 and HCT116 were purchased from ATCC, and cultured according to vendor's instructions. Serum-free conditioned medium from ˜10⁶ cells was collected for downstream iMES assays. Clinical samples (pooled normal plasma, pooled normal urine, individual plasma or urine samples from cancer patients) were purchased from BioIVT (www.biovt.com/). Blood and urine samples were collected from consented donors under IRB-approved protocols, shipped to our laboratory and stored at −80° C. until EV profiling. De-identified clinical information (such as age, gender, ethnicity, diagnosis, disease stage, treatment history, etc) were also provided.

### Immunomagnetic Beads

Five milligrams of magnetic beads coated with epoxy groups (Dynabeads M-270 Epoxy, Invitrogen) was suspended in 0.1 M sodium phosphate solution at room temperature for 10 min. The magnetic beads were separated from the solution with a permanent magnet and resuspended in the same solution. One hundred micrograms of antibodies against CD63 (Ancell), or respective IgG (Biolegend) was added and mixed thoroughly. One hundred microliters of 3 M ammonium sulfate solution were added, and the whole mixture was incubated overnight at 4° C. with slow tilt rotation. The beads were washed twice with PBS solution and finally resuspended in PBS with 1% bovine serum albumin (BSA).

### Biotinylated Antibodies

Sulfo-NHS-biotin (10 mM, Pierce) solution in PBS was incubated with antibodies for 1 hour at room temperature. Unreacted sulfo-NHS-biotin was removed using Zeba spin desalting column, 7K MWCO (Thermo Scientific). Antibodies were kept at 4° C. until use.

### Electronic Sensing Device

The detection device included of a micro-controller (Atmel Corporation; or Texas Instruments), a digital-to-analog converter (Texas Instruments), an analog-to-digital converter (Texas Instruments), a multiplexer (Analog Devices), and eight potentiostats. Each potentiostat included of two operational amplifiers (Analog Devices): one amplifier maintains the potential difference between a working electrode and a reference electrode, and the other one works as a transimpedance amplifier to convert a current to a voltage signal. The gold and carbon electrodes are commercially available (DropSens, Spain).

iMES Assay

10 μL of plasma (or 80 μL of urine, or 80 μL of conditioned medium) was mixed with immunomagnetic bead solution and dilution buffer for 15 minutes at room temperature. Antibodies of interest were mixed with the beads for 15 minutes at room temperature. The magnetic beads were separated from the solution with a permanent magnet, and then re-suspended in dilution buffer. Streptavidin-conjugated HRP enzymes were mixed with the beads for 10 minutes at room temperature. The magnetic beads were separated, magnetically washed, and re-suspended in PBS. The prepared bead solution and UltraTMB solution (ThermoFisher Scientific) were loaded on top of the screen-printed electrode. After 2 minutes, chronoamperometry measurement was started with the electrochemical sensor. The current levels in the range of 50-60 seconds were averaged.

### OTHER EMBODIMENTS

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A method for predicting or monitoring effectiveness of cancer immunotherapy, comprising:
providing a bodily fluid sample from a subject, wherein the bodily fluid sample comprises extracellular vesicles;
analyzing the sample using an integrated magnetic-electrochemical sensing system; and
determining an amount of one or more biomarkers that are associated with the extracellular vesicles, wherein the biomarkers are selected from: MLH1, MSH2, MSH6, PMS2, Perforin, Granzymes, IL-2, IL-6, IL-12, TCF7, and Lin28, and optionally one or more of PD-L1, CD47, and PD-1.

2. (canceled)

3. The method of claim 1, wherein the bodily fluid sample is plasma and/or urine.

4. (canceled)

5. The method of claim 1, wherein the determining step comprises using a SingleMarkerScore method, a CategoryScore method, a RegressionScore method, a DecisionTreeScore method, or a NeuralNetworkScore method.

6. The method of claim 1, wherein the cancer immunotherapy is PD-1/PD-L1, CTLA4, and/or CD47 blockade.

7. The method of claim 1, wherein the cancer is one or more of: bladder, gastric cancer, lung cancer, colon cancer, prostate cancer, renal cell carcinoma, head and neck cancer, melanoma, liver cancer, esophageal cancer, pancreatic cancer, acute myeloid leukemia (AML), chronic myeloid leukemia (CML), glioblastoma, medulloblastoma, oral squamous cell carcinoma, breast cancer, ovarian cancer, cervical cancer, testicular germ cell tumor, and Wilms tumor.

8. A method for diagnosing or prognosing cancer, comprising:
providing a bodily fluid sample from a subject, wherein the bodily fluid sample comprises extracellular vesicles;
analyzing the sample using an integrated magnetic-electrochemical sensing system; and
determining an elevated amount of a biomarkers that are associated with the extracellular vesicles, in comparison with a control, wherein the biomarker is Lin28 and optionally one or more of PD-L1, CD47, and PD-1.

9. The method of claim 8, wherein the bodily fluid sample is plasma and/or urine.

10. (canceled)

11. The method of claim 8, wherein the determining step comprises using a SingleMarkerScore method, a CategoryScore method, a RegressionScore method, a DecisionTreeScore method, or a NeuralNetworkScore method.

12. The method of claim 8, wherein the cancer is one or more of: bladder, gastric cancer, lung cancer, colon cancer, prostate cancer, renal cell carcinoma, head and neck cancer, melanoma, liver cancer, esophageal cancer, pancreatic cancer, acute myeloid leukemia (AML), chronic myeloid leukemia (CML), glioblastoma, medulloblastoma, oral squamous cell carcinoma, breast cancer, ovarian cancer, cervical cancer, testicular germ cell tumor, and Wilms tumor.

13. The method of claim 1, wherein the biomarkers are MLH1, MSH2, PD-L1, and Lin28.

14. The method of claim 1, wherein the biomarkers are MLH1, MSH2, PD-L1, and IL-12.

15. The method of claim 1, wherein the biomarkers are MLH1, MSH2, PD-L1, and TCF7.

16. The method of claim 1, wherein the biomarkers are MLH1, MSH2, PD-L1, perforin, and granzymes.

17. (canceled)

18. The method of claim 1, wherein the biomarkers are MLH1, MSH2, PD-L1, IL-12 and TCF7.

19. (canceled)

20. (canceled)

21. (canceled)

22. The method of claim 1, wherein the biomarkers are PD-L1, CD47, TCF7, and IL-12.

23. (canceled)

24. The method of claim 1, wherein the biomarkers are PD-L1, Perforin, Granzymes, IL-12, and TCF7.

25. The method of claim 1, wherein the biomarkers are MLH1, MSH2, PD-L1, Perforin, Granzymes, IL-12, and TCF7.

26. (canceled)

27. (canceled)

28. The method of claim 1, wherein the biomarkers are MLH1, MSH2, PD-L1, CD47, Perforin, Granzymes, IL-12, and TCF7.

29. The method of claim 1, wherein the biomarkers are PD-L1, CD47, Perforin, Granzymes, IL-12, TCF7, and Lin28.

30. The method of claim 1, wherein the biomarkers are MLH1, MSH2, MSH6, PMS2, PD-L1, CD47, PD-1, Perforin, Granzymes, IL-2, IL-6, IL-12, TCF7, and Lin28.
